(19) United States
(12) Patent Application Publication
(10) Pub. No.: US 2006/0045927 A1
(43) Pub. Date: Mar. 2, 2006

(54) Herbal formulations for modulating blood lipids

(76) Inventors: Dajian Yang, Shenzhen City (CN); Meng Yu Huang, Shenzhen City (CN); Shi Lin Chen, Shenzhen City (CN); Albert Sun-Chi Chan, Hong Kong (HK)

Correspondence Address: KNOBBE MARTENS OLSON & BEAR LLP, 2040 MAIN STREET, FOURTEENTH FLOOR, IRVINE, CA, 92614, US

(21) Appl. No.: 10/925,674

(22) Filed: Aug. 25, 2004

(51) Int. Cl. A61K 36/9062; A61K 36/488

(52) U.S. Cl. 424/756; 424/757

(57) ABSTRACT

The present invention concerns herbal formulations for the modification of the levels of blood lipids. Aspects of the invention include the preparation of herbal formulations and methods for their use.

### FIELD OF THE INVENTION

The present invention relates to plant extracts and their use in pharmaceutical formulations to modify blood lipid levels.

### BACKGROUND OF INVENTION

Alpinia oxyphylla (AO) is a member of the ginger family and is known in Chinese herbal medicine as Yi Zhi Ren. It has a long history as a component of a formula used for centuries to control frequent urination and loss of bladder control. It contains an aromatic oil thought to have anti-inflammatory effects. Alpinia oxyphylla is also used in the treatment of nausea, vomiting and diarrhea. It taste slightly bitter and is warm in nature. It can warm the spleen and inhibit diarrhea and reduce saliva secretion, warm the kidney, control seminal emission and reduce urination. The major function is to treat cold spleen and diarrhea, cold and painful abdomen, excessive saliva, enuresis due to kidney deficiency, frequent urination, turbid spermatorrhea. Because of the many beneficial effects, scientists are interested in finding out other medical uses of AO.

The use of plant extracts to treat medical conditions and diseases is well known in the art. Some raw herbs that have been associated with blood lipid lowering effects included Rhizoma Alismatis, Fructus Crataegi, Ganoderma Lucidum, Radix Polygoni multiflori, Radix Puerariae, Semen Cassiae, Plantula Artemisiae capillaris, Rhizoma Polygoni cuspidati, Pollen Typhae and Bulbus Allii. It is commonly acknowledged in Eastern medical traditions that herbs such as Rhizoma Alismatis, Radix Polygoni multiflori, Rhizoma Polygonati and Fructus Lycii can cure fatty liver. The use of herbs such as Rhizoma Drynariae, Radix Polygoni multiflori, Rhizoma Alismatis, Semen Cassiae, Fructus Crataegi, Ganoderma Lucidum, Pollen Typhae, Dioscorea nipponica Makino, wild rose and oil extracts of Bulbus Allii is purported to have anti-atherosclerotic effects.

Hyperlipidemia (HLP) is a common and widespread condition in middle aged and elderly populations. It has been shown that the occurrence of cerebrovascular diseases is closely related to metabolic disorders of body lipids and changes in the levels of blood lipids.

The comparative and absolute deficiency of insulin in diabetic patients causes disorders in glucose and lipid metabolism and is usually accompanied by hyperlipidemia. The use of herbal medicines or compound formulations of herbs listed in the paragraph above has been shown to lower the blood lipid levels and prevent atherosclerosis in combination with the treatment of diabetes. However, additional or enhanced medicinal effects from the use of other plant extracts or formulations, which may be more powerful than those already known and indeed may be useful as treatments in their own right, await discovery.

### SUMMARY OF INVENTION

Embodiments of the present invention encompass herbal formulations, methods of their production and methods of their use in modifying blood lipid levels and in producing pharmaceutical compositions are taught.

Aspects of the invention include pharmaceutical compositions comprising an effective amount of Alpinia oxyphylla (AO) extract to modify serum lipid levels. Additional aspects feature pharmaceutical compositions comprising an effective amount of Radix Puerariae (RP) extract to modify serum lipid levels and pharmaceutical compositions comprising an effective amount of a mixture of AO and RP extracts to modify serum lipid levels. In some embodiments, the pharmaceutical composition comprising an effective amount of a mixture of AO and RP extracts comprises a mixture with a ratio of AO extract to RP extract ranges from 1:10 to 10:1. In additional embodiments, the ratio of AO extract to RP extract in the mixture ranges from 1:1 to 1:2.

Another aspect of the present invention is a method of producing a pharmaceutical composition with an effective amount of AO extract, comprising grounding AO into powder and extracting AO powder with alcohol featured in other embodiments. Additional embodiments include methods of producing the pharmaceutical composition with an effective amount of AO extract that further comprise filtering and concentrating the AO extract solution to a density of 0.1-5.0 g/m. Preferably, the AO extract solution is concentrated to a density of 0.5-3.0 g/ml. In additional embodiments, the alcohol used in the extraction process is 60-90% (v/v) ethanol. Preferably, the ethanol used is 80% (v/v) ethanol.

Further embodiments of the invention include a method of producing a pharmaceutical composition comprising an effective amount of a mixture of AO and RP extracts to modify serum lipid levels that comprises combining an AO extract and a RP extract. In additional embodiments, the RP extract is produced by grounding RP into powder, extracting RP with alcohol and filtering and concentrating the RP extract such that the ratio of raw herb to the RP extract is about 1:10 by weight. In some embodiments, the alcohol used to extract RP is 20-50% (v/v) ethanol. Preferably, the ethanol used is 30% (v/v). Additional embodiments include methods of treating hyperlipidemia comprising administering an effective amount of AO extract for the modification of serum lipid levels as well as methods of treating hyperlipidemia comprising administering a pharmaceutical composition comprising an effective amount of a mixture of AO and RP extracts for the modification of serum lipid levels. In some embodiments, the ratio of AO extract to RP extract in the mixture ranges from 1:10 to 10:1. In a preferred embodiments, the ratio of AO extract to RP extract in the mixture ranges from 1:1 to 1:2.

Methods of modulating the level of a blood component selected from the group consisting of total cholesterol, triglyceride, HDL-C, and LDL-C comprising the administration of AO extract are featured in additional embodiments of the invention, as are methods of modulating the level of a blood component selected from the group consisting of total cholesterol, triglyceride, HDL-C, and LDL-C comprising the administration of a mixture of AO extract and RP extract. In some embodiments, the ratio of AO extract to RP extract in the mixture ranges from 1:10 to 10:1. In a preferred embodiment, the ratio of AO extract to RP extract in the mixture ranges from 1:1 to 1:2.

Additional embodiments feature methods of preparing a pharmaceutical composition for the modulation of the level of a blood component selected from the group consisting of total cholesterol, triglyceride, HDL-C, and LDL-C, comprising the incorporation of AO extract into the composition, as well as methods of preparing a pharmaceutical composition for the modulation of the level of a blood component selected from the group consisting of total cholesterol, triglyceride, HDL-C, and LDL-C, comprising the incorporation of a mixture of AO extract and RP extract into the composition. In some embodiments, the ratio of AO extract to RP extract in the mixture ranges from 1:10 to 10:1. In some embodiments, the ratio of AO extract to RP extract in the mixture ranges from 1:1 to 1:2.

A process for obtaining a RP extract is included in additional embodiments, comprising grinding RP, extracting ground RP with alcohol, filtering and concentrating the extract solution to create an extract filtrate, purifying the extract filtrate and drying the extract filtrate to produce a RP extract in powder form. In some embodiments, the ground RP is extracted with 30% (v/v) ethanol. In some embodiments, the extraction of ground RP with alcohol is performed three times for one hour per extraction. Additional embodiments of the method include purifying of the extract filtrate with a resin column, drying of the extract filtrate under a vacuum and/or combining the RP extract with a pharmaceutically acceptible carrier.

A process for obtaining an AO extract is included in additional embodiments, comprising grinding AO, extracting ground AO with alcohol, filtering and concentrating the extract solution to create a concentrated AO extract solution, adding stabilizing agents to the concentrated AO extract solution to create an AO mixture, and drying the AO mixture to produce an AO extract. In some embodiments, the ground AO is extracted with 80% (v/v) ethanol. In some embodiments, the extraction of ground AO with alcohol is performed three times for one hour per extraction. Additional embodiments feature stabilizing agents selected from the group consisting of lactose and silica gel and/or the step of combining the AO extract with a pharmaceutically acceptable carrier.

A process for obtaining a mixture of AO and RP extracts is included in additional embodiments, comprising combining an AO extract, a RP extract and stabilizing agents, grinding the extract and stabilizing agent combination and granulating the ground combination to create a mixture of AO and RP extracts. In some embodiments, the process includes a step of combining the mixture with a pharmaceutically acceptable carrier. In some embodiments, a pharmaceutical composition for the modulation of blood lipid levels is obtained by encapsulating the mixture of AO and RP extracts.

### BRIEF DESCRIPTION OF THE DRAWINGS

FIG. 1 shows a flow chart detailing the production of supplement capsules containing material from A. oxyphylla (AO) and Radix Puerariae (RP). The procedure is described below in Example 1 of the Detailed Description.

### DETAILED DESCRIPTION

Embodiments of the invention are based in the surprising discovery of the utility of Alpinia oxyphylla in the treatment of altered blood lipid levels, a heretofore unknown property of the plant. Extracts of Alpinia oxyphylla (AO) show strong effects on the modulation of cholesterol and triglyceride levels, and in combination with raw herbs known to modulate blood lipids, the comprehensive modulation effect of blood lipids can be increased. Accordingly, aspects of the invention include the preparation of blood lipid-modulating pharmaceuticals. Additional aspects of the invention include method of providing blood lipid modulating effects that utilize AO or some fraction of AO. Other aspects of the invention concern products of high efficacy in the regulation of blood lipid levels.

Particular embodiments of the invention involve the use of Alpinia oxyphylla in preparing a pharmaceutical composition for modulating blood lipids levels. Aspects of the present invention include the use of Alpinia oxyphylla for the treatment of blood lipid disorders and cardiovascular diseases. Additional aspects include the use of Alpinia oxyphylla to create extracts and medicaments for the treatment of blood lipid disorders and cardiovascular diseases. Furthermore, some embodiments take advantage of the surprising discovery that Alpinia oxyphylla in combination with Radix Puerariae (RP) produces an enhanced blood lipid-modulating effect as compared to the use of Alpinia oxyphylla or Radix Puerariae alone.

For oral administration, the extracts may be provided as a tablet, aqueous or oil suspension, dispersible powder or granule, emulsion, hard or soft capsule, syrup, elixir, or beverage. Compositions intended for oral use may be prepared according to any method known in the art for the manufacture of pharmaceutically acceptable compositions and such compositions may contain one or more of the following agents: sweeteners, flavoring agents, coloring agents and preservatives. The sweetening and flavoring agents will increase the palatability of the preparation. Tablets containing extracts in admixture with non-toxic pharmaceutically acceptable excipients suitable for tablet manufacture are acceptable. Pharmaceutically acceptable means that the agent should be acceptable in the sense of being compatible with the other ingredients of the formulation (as well as non-injurious to the patient). Such excipients include inert diluents such as calcium carbonate, sodium carbonate, lactose, calcium phosphate or sodium phosphate; granulating and disintegrating agents, such as corn starch or alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl distearate alone or with a wax may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil. In some embodiments, aqueous suspensions may contain an extract of the invention in admixture with excipients suitable for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as arachis oil, olive oil, sesame oil or coconut oil, or in a mineral oil such as liquid paraffin. The oil suspension may contain a thickening agent, such as beeswax, hard paraffin or acetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide one or more extracts in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

The extract preparations for parenteral administration may be in the form of a sterile injectable preparation, such as a sterile injectable aqueous or oleaginous suspension. This suspension may be formulated according to methods well known in the art using suitable dispersing or wetting agents and suspending agents. The sterile injectable preparation may also be a sterile injectable solution or suspension in a non-toxic parenterally-acceptable diluent or solvent, such as a solution in 1,3-butanediol. Suitable diluents include, for example, water, Ringer's solution and isotonic sodium chloride solution. In addition, sterile fixed oils may be employed conventionally as a solvent or suspending medium. For this purpose, any bland fixed oil may be employed including synthetic mono or diglycerides. In addition, fatty acids such as oleic acid may likewise be used in the preparation of injectable preparations.

The pharmaceutical compositions may also be in the form of oil-in-water emulsions. The oily phase may be a vegetable oil, such as olive oil or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

The amount of extract that may be combined with the carrier material to produce a single dosage form will vary depending upon the host treated and the particular mode of administration.

As used in the present specification and claims, the terms “comprise,” “comprises,” and “comprising” mean “including, but not necessarily limited to.” For example, a method, apparatus, molecule or other item which contains A, B, and C may be accurately said to comprise A and B. Likewise, a method, apparatus, molecule or other item which “comprises A and B” may include any number of additional steps, components, atoms or other items as well.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of that which is described and claimed.

### EXAMPLES

The following examples teach the methods and compositions disclosed herein for modulating blood lipid levels through the administration of extracts of plants. These examples are illustrative only and are not intended to limit the scope of the invention disclosed herein. The treatment method described below can be optimized using empirical techniques well known to those of ordinary skill in the art. Moreover, artisans of skill would be able to use the teachings described in the following examples to practice the full scope of the invention disclosed herein.

### Example 1

### Preparation of a Blood Lipid-Modulating Supplement Capsule

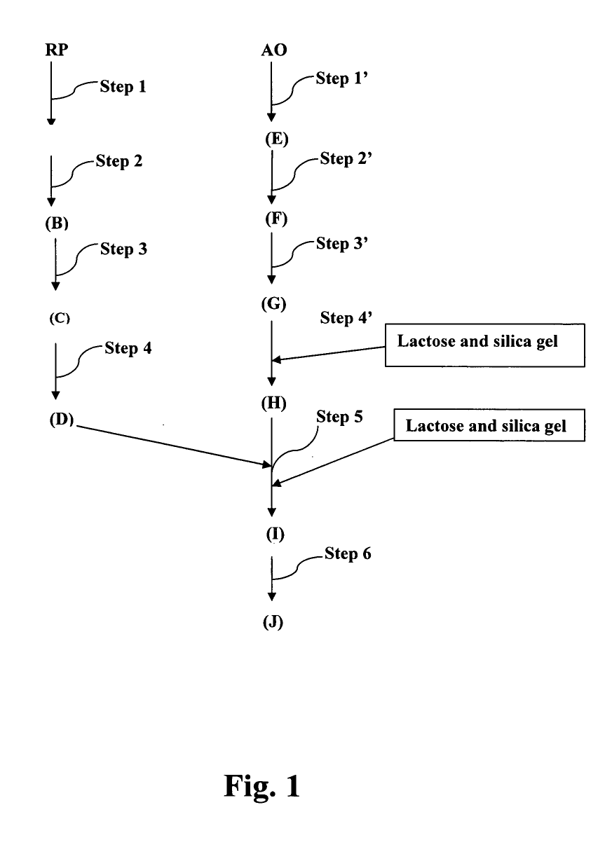

The process of preparing RP, AO, RP+AO extracts are described with reference to FIG. 1.

1.1 Ingredients

Capsules were manufactured using 2000 g of Radix Puerariae, 1000 g of A. oxyphylla, 114 g of lactose and 30 g of silica gel according to the instructions below. These raw materials and excipients produced 1000 capsules of size 0#.

1.2 Method

In step 1, 2000 g of RP was grounded into coarse powder (A). In step 2, the coarse powder of RP (A) was extracted by adding 7 folds by weight of 30% (v/v) ethanol. The extraction was repeated for 3 times, one hour per extraction. The three extractions were then pooled to obtain the RP extract solution (B). The RP extract solution (B) obtained was filtered and concentrated under reduced pressure in step 3 to obtain RP filtrate (C). The concentration of the RP filtrate (C) was then adjusted to a ratio of 1:10 (weight ratio) of raw herbs to filtrate. The separation and purification was carried out in step 4 by the use of resin column AB-8 (Source: The Chemical Factory of Nankai University, Tianjin, PRC). Sample loading was carried out at the flow rate of 1 bed volume (BV)/h. The volume of sample did not exceed 1.4 BV. The column was washed with 2 BV of water, followed by elution with 4 BV of 70% ethanol at the flow rate of 2 BV/h. The fractions eluted by 70% (v/v) ethanolwere pooled, concentrated under reduced pressure and then dried by vacuum drying to obtain 180 g of RP powder (D).

In step 1′, 1000 g of AO was grounded into a coarse powder (E). In step 2′, the coarse powder of AO (E) was extracted with 5 folds by weight of 80% ethanol. The extraction was repeated 3 times, one hour per extraction. The three extractions were then pooled to obtain 2.5 kg of the AO extract solution (F). The extract solution (F) thus obtained was filtered and concentrated under reduced pressure in step 3′ to obtain 160 g of concentrated AO extract (G) with a relative density of 1.35-1.38 g/ml (detected at 55° C.). In step 4′, approximately 85.5 g of lactose and 22.5 g of silica gel were added to the concentrated AO extract (G) to form the AO mixture (H) and the mixture was dried at 60° C. in a ventilating oven.

Capsules containing RP+AO were created from step 5. The AO mixture (H) was cooled to below 0° C. 28.5 g of lactose and 7.5 g of silica gel as well as the 180 g of dried RP powder (D), were added to form 450 g of AO+RP mixture (I). In step 6, the AO+RP mixture (I) was further grounded and passed through the 60-mesh sieve. Granulation was carried out by the use of 95% (v/v) ethanol as an adhesive. The granules were dried at 60° C. and encapsulated to produce the capsules (J).

### Example 2

### Study of the Blood Lipid Modulating Effect of Formulations Containing AO Extract on Acute Hyperlipidemia in Mice using Yolk Emulsion Model

The use of intraperitoneal injection of yolk emulsion is to induce acute hyperlipidemia in the mice. The advantage of this experiment is the animal model can be established in a relatively short time (Xu S. Y. etc. eds. Pharmacology Experimental Methodology. People's Hygiene Press. 200x: 1202-1203). The use of egg emulsion mainly induces the rise in cholesterol but not in triglyceride.

2.1 Materials

2.1.1 Pharmaceuticals and Reagents:

For the RP extract, 1.3824 g of RP powder (D) was combined with sterile water to create 24 ml of RP extract, with a concentration of 0.0576 g/ml. For the AO extract, 0.6048 g of concentrated AO extract (G) was combined with sterile water and a drop of Tween 80 to make up 24 ml of AO extract, with a concentration of 0.0252 g/ml. Tween 80 was added to facilitate the dissolution of the concentrated AO extract in water.

For the RP+AO extract, 0.649 g of (D) was combined with sterile water to make up 12 ml of extract. 0.577 g of (G) was combined with sterile water and a drop of Tween 80 to make up 12 ml of extract. The two extracts were combined by vortex mixing.

For samples of evening primrose oil, 1.6000 g of evening primrose oil from capsules (Guangzhou Xinqun Pharmaceuticals Joint Stock Company, batch no: 0208001) was combined with sterile water to make up 24 ml of oil-water mixture, with a concentration of 0.0667 g/ml. The mixture was vortexed to form emulsion.

Testing kits with reagents for total cholesterol (TC) were purchased from Zhongsheng Beikong Biotechnology Joint Stock Company, batch no: 180051. Testing kits with reagents for serum triglycerides (TG) were purchased from Zhongsheng Beikong Biotechnology Joint Stock Company, batch no: 220241. Testing kits with reagents for high density lipoprotein cholesterol (HDL-C) were purchased from Zhongsheng Beikong Biotechnology Joint Stock Company, batch no: 190031.

2.1.2 Major Equipment

A 5415D benchtop centrifuge (Eppendorf) and a XD811 clinical analyzer (Shanghai Xunda Medical Equipment Ltd.) were used in the experiments.

2.1.3 Animals

Sixty SPF grade KM mice, 18-22 g, both males and females, were supplied by the Guangzhou Chinese Medicine Laboratory Animals Centre, Certificate No: 2002A005 (Yue Jian and Zheng Zi), Quality Certificate provided by the Institute of Monitoring of Laboratory Animals of the Guangdong Province Technology Committee.

2.2 Methods

2.2.1 Grouping

The mice were randomly separated into 6 groups. One group received no yolk emulsion and no extracts (“Normal” group). One group received the yolk emulsion without the administration of any extract and was named the Model group (negative control group). The other four groups received RP extract (RP group), AO extract (AO group), AO+RP extract (AO+RP group), or evening primrose oil (positive control group), respectively, in addition to the yolk emulsion.

2.2.2 Procedures

Fresh chicken egg yolks were added with physiologically isotonic saline to make 75% yolk emulsion.

For Normal group, the mice received neither treatment nor injections of yolk emulsion.

For Model group (negative control) group, each mouse was fasted for 16 hours before giving intraperitoneal (i. p.) injections of 0.5 ml of the yolk emulsion.

The mice in RP group, AO group, AO+RP group and the positive control group were given the corresponding extract via intragastric gavage (i.g.) once per day for 3 consecutive days. The amount of extract administered to the mice was 0.1 ml/10 g body weight. On the third day, 1 hour after treatment, injections (i. p.) of 75% yolk emulsion was given to the mice. Three hours after injection, the mice were given corresponding extract via the same treatment once more.

Twenty hours after injection, blood was collected by retro orbital bleeding technique from the eye socket from the mice in each group and then centrifuged for 10 minutes at 3000 rpm. Serum from the blood samples was analyzed to determine the concentrations of TC, TG and HDL-C.

2.3 Results

The figures were shown in the form of x±s and t testing was carried out.

TABLE 1
The effectiveness of AO extracts, RP extracts and the mixture thereof on
hyperlipidemia in mice using Yolk Emulsion Model.

| | | | Result | | |
|---|---|---|---|---|---|
| | No. of | Dosage | TC | TG | HDL-C |
| Group | animals | (g/kg) | (mmol/L) | (mmol/L) | (mmol/L) |
| Normal | 10 | 0 | 4.45 ± 2.48** | 1.40 ± 0.34 | 1.52 ± 0.29 |
| Model | 10 | 0 | 7.45 ± 1.66 | 1.17 ± 0.37*** | 1.32 ± 0.37 |
| AO | 10 | 0.252 | 4.75 ± 0.95** | 1.43 ± 0.39 | 1.77 ± 0.26**^{▴} |
| RP | 10 | 0.576 | 4.78 ± 1.73** | 1.06 ± 0.22^{▴▴} | 2.08 ± 0.95**^{▴} |
| AO + RP | 10 | 0.511 | 4.64 ± 1.63** | 1.06 ± 0.49^{▴} | 1.77 ± 0.31**^{▴} |
| Positive control | 10 | 0.667 | 4.47 ± 0.78** | 1.24 ± 0.51 | 1.83 ± 0.66* |

*p < 0.05 compared to model group;
**p < 0.01 compared to model group
^{▴}p < 0.05 compared to normal group;
^{▴▴}p < 0.01 compared to normal group
***The injection yolk emulsion mainly increases the level of cholesterol and its primary effect is not on TG.

The results showed that the TC level of the mice in normal group and other treatment groups was significantly lower than that of the model group (p<0.01). None the treatment groups showed significant differences as compared to the normal group.

The HDL-C level of the mice in the normal group and other treatment groups was significantly higher than that that in the model group. There was a very significant difference in levels of HDL-C (p<0.01) between mice in the groups receiving AO, RP or AO+RP extracts and mice in the model group. The mice in the evening primrose oil group also showed significant differences compared to the model group (p<0.05). When compared to normal group, the mice in the AO extract group, RP extract group and the AO+RP extract group showed a significant increase in the HDL-C level (p<0.05).

### Example 3

### Study of the Blood Lipids Modulating Effect of the Compound Formulation Containing AO, RP or a Mixture thereof on Hyperlipidemia in Rats using Hyperlipedmia Model

3.1 Materials

3.1.1 Pharmaceuticals and Reagents:

For the RP extract, 0.1728 g of RP powder (D) was combined with sterile water to make up 3.0 ml of RP extract, with a concentration of 0.0576 g/ml. For the AO extract: 0.0756 g of concentrated AO extract (G) was combined with sterile water and a drop of Tween 80 to make up 3.0 ml of AO extract, with a concentration of 0.0252 g/ml (Tween 80 was added as a dispersant). To create the combined RP+AO extract, 0.649 g of (D) was combined with sterile water to make up 12 ml of extract, 0.577 g of (G) was combined with sterile water and a drop of Tween 80 to make up 12 ml of extract and the two extracts were combined by vortex mixing.

For evening primrose oil, 0.2000 g of evening primrose oil from capsules (purchased from Guangzhou Xinqun Pharmaceuticals Joint Stock Company, batch no: 0208001) was combined with sterile water to make up 3.0 ml of oil-water mixture, with a concentration of 0.0667 g/ml. The mixture was vortexed to give a emulsion.

Cholesterol was purchased from Beijing Dingguo Biotechnology Development Centre, sodium deoxycholate was purchased from China Pharmaceuticals Group Shanghai Chemical Reagent Ltd. (F20021218), propycil was purchased from Guangzhou Shiqiao Pharmaceuticals Company Ltd. (batch no: 020601) and testing kits for LDL-C with reagents were purchased from Zhongsheng Beikong Biotechnology Joint Stock Company (batch no: 020604).

To create the hyperlipidemia model, a lipid emulsion was prepared for use in the experiments described below. The emulsion was prepared by adding 10% cholesterol, 20% lard, 2% sodium deoxycholate and 1% of propycil to water. The emulsion was formed by continuous stirring in a water bath at 37° C. during the administration to the rats. The percentage is a w/v percentage, i.e. 1 g in 100 ml of emulsion is considered as 1%.

3.1.2 Major Equipment

5415D benchtop centrifuge (Eppendorf) and XD811 Clinical Analyzer (Shanghai Xunda Medical Equipment Company).

3.1.3 Animals

Sixty SPF grade SD rats, 180-220 g, of both sexes, supplied by the First Military University Laboratory Animal Centre, Certificate No: 2002A005 (Yue Jian Zheng Zi). Quality Certificate provided by the Institute of Monitoring of Laboratory Animals of the Guangdong Province Technology Committee.

3.2 Methods

3.2.1 Grouping:

One group received no injections and was named the Normal group. One group received the lipid emulsion without administering any extract and was named the Model group (negative control group). The other four groups received the lipid emulsion before administration of the RP extract, AO extract, AO+RP extract or evening primrose oil (positive control group), respectively.

The emulsion was given to rats by forced feeding, 0.4 ml/200 g body weight, for consecutive 7 days. The hyperlipemia modeling and the various treatment were started on the same day and continued for the consecutive 7 day period. On day 8, blood was collected by retro orbital bleeding technique from the eye socket, and was centrifuged at 3000 rpm for 10 minutes to obtain serum samples. Measurements of the concentrations of TC, TG, HDL-C and LDL-C in the serum samples were taken.

3.3 Results

TABLE 2
The effectiveness of AO extracts, RP extracts and the mixture thereof on
hyperlipidemia in mice using Hyperlipemia Model

| | | | Results | | | |
|---|---|---|---|---|---|---|
| | No of | Dosage | TC | TG | HDL-C | LDL-C |
| Group | animals | (g/kg) | (mmol/L) | (mmol/L) | (mmol/L) | (mmol/L) |
| Normal | 10 | 0 | 1.57 ± 0.32** | 0.74 ± 0.26 | 0.91 ± 0.17 | 1.85 ± 0.33** |
| Model | 10 | 0 | 2.26 ± 0.57 | 1.33 ± 1.41 | 1.04 ± 0.20 | 2.72 ± 0.38 |
| AO | 10 | 0.252 | 1.79 ± 0.26* | 0.52 ± 0.08* | 1.51 ± 0.43**^{▴▴} | 2.31 ± 0.57* |
| RP | 10 | 0.576 | 1.07 ± 0.22**^{▴▴} | 0.57 ± 0.18 | 0.94 ± 0.17 | 1.56 ± 0.38** |
| AO + RP | 10 | 0.511 | 1.49 ± 0.22** | 0.97 ± 0.93 | 1.22 ± 0.22**^{▴▴} | 1.98 ± 0.43** |
| Positive | 10 | 0.667 | 1.09 ± 0.22* | 0.79 ± 0.48 | 1.21 ± 0.35*^{▴} | 2.61 ± 0.43 |
| control | | | | | | |

*p < 0.05 compared to model group;
**p < 0.01 compared to model group
^{▴}p < 0.05 compared to normal group;
^{▴▴}p < 0.01 compared to normal group

The results showed that TC levels in serum from rats in the normal group and the other treatment groups were all lower than that of the model group. The TC levels in serum from rats in the normal, RP extract and AO+RP extract groups were very significantly lower than those of the model group (p<0.01). The TC levels in serum from rats of the AO extract and evening primrose oil groups were significantly lower than those of the model group (p<0.05). When compared to normal group, the TC levels in serum from rats in the RP group were very significantly lower (p<0.01) than those of the model group.

The TG levels in serum from rats in the normal group and the other treatment groups were lower than that of the model group, but only the serum from rats in the AO extract group showed a significant difference (p<0.05) as compared to serum from the model group.

The serum HDL-C levels in rats from the AO extract, AO+RP extract and the evening primrose oil groups were all higher than that of the model group. Furthermore, the AO extract and AO+RP extract groups showed very significant differences as compared to the model group (p<0.01), and the evening primrose oil group showed a significant difference compared to the model group (p<0.05). When compared to the normal group, the AO and AO+RP extract groups showed very significant differences (p<0.01). The evening primrose oil group showed a significant difference (p<0.05) when compared to the normal group.

When serum LDL-C levels were examined, the normal group and all treatment groups except the evening primrose group were found to have significantly lower levels than the model group. The normal, RP extract and AO+RP extract groups all showed very significant differences as compared to the model group (p<0.01).

Evaluations of the comprehensive blood lipid modulation effects of the AO extract and the AO+RP extract are shown in Table 3.

TABLE 3
The evaluation of the comprehensive blood lipid modulation effect of the
AO extract, the RP extract, and the AO + RP mixture.

| | Hyperlipidemic | | | Hyperlipidemic | | | |
|---|---|---|---|---|---|---|---|
| | mice | | | rats | | | |
| Group | TC | TG | HDL | TC | TG | HDL-C | LDL-C |
| AO | ** | | **▴ | * | * | **▴▴ | * |
| RP | ** | ▴▴ | **▴ | **▴▴ | | | ** |
| AO + RP | ** | ▴ | **▴ | ** | | **▴▴ | ** |
| Positive control | ** | | * | * | | *▴ | |

*p < 0.05 compared to model group;
**p < 0.01 compared to model group
^{▴}p < 0.05 compared to normal group;
^{▴▴}p < 0.01 compared to normal group

The table shows that the AO extract, the RP extract, evening primrose oil and the AO+RP extract mixture all possessed the blood lipid modulation effect and that the AO+RP extract mixture showed the best comprehensive blood lipid modulation effect. The AO extract by itself is also very effective in modulating blood lipid levels and is able to support blood lipid modulation in compound formulation.

While the preferred embodiment of the invention has been illustrated and described, it will be appreciated that various changes can be made therein by the one skilled in the art without departing from the spirit and scope of the invention. Accordingly, the scope of this invention is intended to be defined only by reference to the appended claims.

1. A pharmaceutical composition comprising an effective amount of AO extract to modify serum lipid levels.

2. A pharmaceutical composition comprising an effective amount of Radix Puerariae (RP) extract to modify serum lipid levels.

3. A pharmaceutical composition comprising an effective amount of a mixture of AO and RP extracts to modify serum lipid levels.

4. The pharmaceutical composition according to claim 3, wherein the ratio of AO extract to RP extract in said mixture ranges from 1:10 to 10:1.

5. The pharmaceutical composition according to claim 4, wherein the ratio of AO extract to RP extract in said mixture ranges from 1:1 to 1:2.

6. A method of producing the pharmaceutical composition according to claim 1, comprising
grounding AO into powder; and
extracting AO powder with alcohol.

7. The method according to claim 6, further comprising filtering and concentrating the AO extract solution to a density of 0.5-3.0 g/ml.

8. The method according to claim 6, wherein the alcohol is 80% ethanol.

9. A method of producing a pharmaceutical composition according to claim 3, comprising combining an AO extract and a RP extract.

10. The method according to claim 9, wherein the RP extract is produced by grounding RP into a powder;
extracting RP with alcohol; and
filtering and concentrating the RP.

11. The method according to claim 10, wherein the alcohol is 30% (v/v) ethanol.

12. A method of treating hyperlipidemia comprising administering an effective amount of AO extract for the modification of serum lipid levels.

13. A method of treating hyperlipidemia comprising administering a pharmaceutical composition comprising an effective amount of a mixture of AO and RP extracts for the modification of serum lipid levels.

14. The method according to claim 13, wherein the ratio of AO extract to the RP extract in said mixture ranges from 1:10 to 10:1.

15. A method of modulating the level of a blood component selected from the group consisting of total cholesterol, triglyceride, HDL-C, and LDL-C, comprising the administration of AO extract.

16. A method of modulating the level of a blood component selected from the group consisting of total cholesterol, triglyceride, HDL-C, and LDL-C, comprising the administration of a mixture of AO extract and RP extract.

17. The method of claim 16, wherein the ratio of AO extract to RP extract in said mixture ranges from 1:10 to 10:1.

18. A method of preparing a pharmaceutical composition for the modulation of the level of a blood component selected from the group consisting of lipids, total cholesterol, triglyceride, HDL-C, and LDL-C, comprising the incorporation of AO extract into said composition.

19. A method of preparing a pharmaceutical composition for the modulation of the level of a blood component selected from the group consisting of lipids, total cholesterol, triglyceride, HDL-C, and LDL-C, comprising the incorporation of a mixture of AO extract and RP extract into said composition.

20. The method of claim 19, wherein the ratio of AO extract to RP extract in the mixture ranges from 1:10 to 10:1.

21. The method of claim 20, wherein the ratio of AO extract to RP extract in the mixture ranges from 1:1 to 1:2.

22. A RP extract obtained by a process comprising:
grinding RP;
extracting ground RP with alcohol;
filtering and concentrating the extract solution to create an extract filtrate;
purifying the extract filtrate; and
drying the extract filtrate to produce a RP extract in powder form.

23. The process of claim 22, wherein the ground RP is extracted with 30% (v/v) ethanol.

24. The process of claim 22, wherein the extraction of ground RP with alcohol is performed three times for one hour per extraction.

25. The process of claim 22, wherein said purifying of the extract filtrate is performed with a resin column.

26. The process of claim 22, wherein said drying of the extract filtrate is performed under a vacuum.

27. The process of claim 22, further comprising the step of combining the RP extract with a pharmaceutically acceptable carrier.

28. An AO extract obtained by a process comprising:
grinding AO;
extracting ground AO with alcohol;
filtering and concentrating the extract solution to create a concentrated AO extract solution;
adding stabilizing agents to the concentrated AO extract solution to create an AO mixture; and
drying the AO mixture to produce an AO extract.

29. The process of claim 28, wherein the ground AO is extracted with 80% (v/v) ethanol.

30. The process of claim 28, wherein the extraction of ground AO with alcohol is performed three times for one hour per extraction.

31. The process of claim 28, wherein said stabilizing agents are selected from the group consisting of lactose and silica gel.

32. The process of claim 28, further comprising the step of combining the AO extract with a pharmaceutically acceptable carrier.

33. A mixture of AO and RP extracts obtained by a process comprising:
combining an AO extract, a RP extract and stabilizing agents;
grinding the extract and stabilizing agent combination; and
granulating the ground combination to create said mixture.

34. The process of claim 33, further comprising the step of combining the mixture with a pharmaceutically acceptable carrier.

35. A pharmaceutical composition for the modulation of blood lipid levels obtained by a process comprising encapsulating the mixture of claim 34.
